(19) United States
(12) Patent Application Publication
(10) Pub. No.: US 2007/0066724 A1
(43) Pub. Date: Mar. 22, 2007

(54) Bisazoquinolone pigments, processes for their preparation and their use

(76) Inventors: Johannes Benkhoff, Basel (CH); Max Hugin, Runenberg (CH); Thomas Eichenberger, Basel (CH)

Correspondence Address: CIBA SPECIALTY CHEMICALS CORPORATION;PATENT DEPARTMENT, 540 WHITE PLAINS RD, P O BOX 2005, TARRYTOWN, NY, 10591-9005, US

(21) Appl. No.: 10/557,224

(22) PCT Filed: Jun. 4, 2004
(86) PCT No.: PCT/EP04/51023

§ 371 Date: Nov. 17, 2005

(30) Foreign Application Priority Data
Jun. 13, 2003 (CH) 2003 1036/03

(51) Int. Cl. C08K 5/34; C09D 11/00; C07D 401/02

(52) U.S. Cl. 524/90; 106/31.77; 534/733

(57) ABSTRACT

Bisazoquinolone pigments which, in one of the tautomeric forms thereof, correspond to formula (1) wherein W is the radical of an unsubstituted or substituted C₆-C₂₄aryl or the radical of an unsubstituted or substituted heteroaryl, Ar₁ is unsubstituted or substituted C₆-C₂₄aryl or unsubstituted or substituted heteroaryl, Ar₁₁ is unsubstituted or substituted C₆-C₂₄aryl or unsubstituted or substituted heteroaryl, R₁ R₁, R₂, R₁₀, R₁₁, and R₁₂ are each independently of the others hydrogen, C₁-C₆alkyl, halogen, cyano, CF₃, nitro, NR₃R₄, COOR₄, NR₄COR₃, COOX⁺, COR₄, OR₄, SR₃, S0₂R₃, S0₂NR₃R₄, S0₃-X⁺, or C₆-C₂₄aryl unsubstituted or mono- or poly-substituted by R₅, R₃ is C₁-C₆alkyl, or C₆-C₁₂aryl unsubstituted or mono- or polysubstituted by halogen, hydroxy, OR₇, cyano, nitro, SR₇, NR₆R₇, COOR₇, CONR₆R₇, NR₆COR₇, NR₆COOR₇, COO—X⁺, COR₄, OR₄, S0₂R₇, S0₂NR₆R₇, S0₃-X⁺ or by S0₃R₇, R₄ is hydrogen or has the same meanings as R₃, R₅ is hydrogen, C₁-C₄alkyl, halogen, nitro, NR₇R₈ or OR₇, R₆ is hydrogen or C₁-C₃alkyl, R₇ and R₈ are each independently of the other hydrogen, C₁-C₃alkyl, phenyl unsubstituted or mono- or poly-substituted by halogen, nitro, OR₅ or by NR₁₆R₁₇, or benzyl unsubstituted or mono- or poly-substituted by halogen, nitro, OR₅ or by NR₁₆R₁₇, and X⁺ is a cation H⁺, Li⁺, Na⁺, K⁺, Mg⁺+_{1/2}, Ca⁺⁺_{1/2}, Sr⁺⁺_{1/2}, Ba⁺⁺_{1/2}, Cu⁺, Cu⁺⁺_{1/2}, Zn⁺⁺_{1/2}, Mn⁺⁺_{1/2}, Al⁺⁺⁺_{1/3} or [NR₁₉R₂₀R₂₁R₂₂]⁺ wherein R₁₉, R₂₀, R₂₁ and R₂₂ are each independently of the others hydrogen, C₁-C₆alkyl, phenyl unsubstituted or mono- or polysubstituted by C₁-C₆alkyl, halogen, nitro, OR₅ or by NR₁₆R₁₇, or benzyl unsubstituted or mono- or poly-substituted by C₁-C₆alkyl, halogen, nitro, OR₅ or by NR₁₆R₁₇, R₁₆ and R₁₇ are each independently of the other hydrogen or C₁-C₆alkyl, Z₁, is —NH— or -0-, and Z₂ is —NH or -0- are suitable for colouring high molecular weight material and are distinguished by the resulting colorations having good fastness properties.

The present invention relates to novel bisazoquinolone pigments, to processes for their preparation and to their use as colorants, especially in the colouring of high molecular weight material.

The ever greater demands made of the quality of colorations, for example their fastness properties, or of application-related properties, for example their migration behaviour or their oversprayability, have resulted in there still being a need for new pigments that exhibit improved properties, especially in respect of fastness properties.

Accordingly, the present invention is based on the problem of finding novel bisazoquinolone pigments, especially for producing surface coatings, printing inks and colour filters or for the colouring of plastics, which pigments possess the above-described qualities to a high degree. The novel pigments should yield colorations having a high purity of shade, high tinctorial strength and good fastness to overspraying and to migration. The resulting colorations should especially exhibit good fastness to heat, to light and to weathering.

It has been found that the problem posed is largely solved using the novel bisazoquinolone pigments defined hereinbelow.

The present invention accordingly relates to bisazoquinolone pigments which, in one of the tautomeric forms thereof, correspond to formula 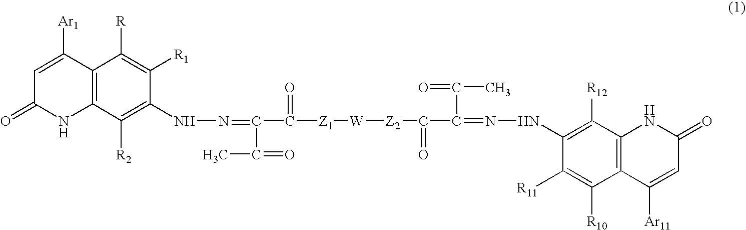
wherein
W is the radical of an unsubstituted or substituted C₆-C₂₄aryl or the radical of an unsubstituted or substituted heteroaryl,
Ar₁ is unsubstituted or substituted C₆-C₂₄aryl or unsubstituted or substituted heteroaryl,
Ar₁₁ is unsubstituted or substituted C₆-C₂₄aryl or unsubstituted or substituted heteroaryl,
R, R₁, R₂, R₁₀, R₁₁ and R₁₂ are each independently of the others hydrogen, C₁-C₆alkyl, halogen, cyano, CF₃, nitro, NR₃R₄, COOR₄, NR₄COR₃, COO^{-X}⁺, COR₄, OR₄, SR₃, SO₂R₃, SO₂NR₃R₄, SO₃⁻X⁺, or C₆-C₂₄aryl unsubstituted or mono- or poly-substituted by R₅,
R₃ is C₁-C₆alkyl, or C₆-C₁₂aryl unsubstituted or mono- or poly-substituted by halogen, hydroxy, OR₇, cyano, nitro, SR₇, NR₆R₇, COOR₇, CONR₆R₇, NR₆COR₇, NR₆COOR₇, COO⁻X⁺, COR₄, OR₄, SO₂R₇, SO₂NR₆R₇, SO₃⁻X⁺ or by SO₃R₇,
R₄ is hydrogen or has the same meanings as R₃,
R₅ is hydrogen, C₁-C₄alkyl, halogen, nitro, NR₇R₈ or OR₇,
R₆ is hydrogen or C₁-C₃alkyl,
R₇ and R₈ are each independently of the other hydrogen, C₁-C₃alkyl, phenyl unsubstituted or mono- or poly-substituted by halogen, nitro, OR₅ or by NR₁₆R₁₇, or benzyl unsubstituted or mono- or poly-substituted by halogen, nitro, OR₅ or by NR₁₆R₁₇, and X⁺ is a cation H⁺, Li⁺, Na⁺, K⁺, Mg⁺⁺_{1/2}, Ca⁺⁺_{1/2}, Sr⁺⁺_{1/2}, Ba⁺⁺_{1/2}, Cu⁺, Cu⁺⁺_{1/2}, Zn⁺⁺_{1/2}, Mn⁺⁺_{1/2}, Al⁺⁺⁺^{1/3} or
[NR₁₉R₂₀R₂₁R₂₂]⁺ wherein R₁₉, R₂₀, R₂₁ and R₂₂ are each independently of the others hydrogen, C₁-C₆ alkyl, phenyl unsubstituted or mono- or poly-substituted by C₁-C₆alkyl, halogen, nitro, OR₅ or by NR₁₆R₁₇, or benzyl unsubstituted or mono- or poly-substituted by C₁-C₆alkyl, halogen, nitro, OR₅ or by NR₁₆R₁₇,
R₁₆ and R₁₇ are each independently of the other hydrogen or C₁-C₆alkyl,
Z₁ is —NH— or —O—, and
Z₂ is —NH— or —O—.

As C₆-C₂₄aryl there come into consideration for W, Ar₁ and Ar₁₁ each independently of the others e.g. phenyl, 1-naphthyl, 2-naphthyl, 4-biphenyl, phenanthryl, 2- or 9-fluorenyl or anthryl, especially phenyl, 1-naphthyl or 2-naphthyl.

W, Ar₁ and Ar₁₁ as heteroaryl are each independently of the others, for example, a polyunsaturated heterocyclic structure having from 5 to 18 atoms selected from C, N, O and S, which contains at least 6 conjugated π-electrons. Such a structure is, for example, thienyl, benzo[b]thienyl, dibenzo[b,d]thienyl, thianthrenyl, furyl, furfuryl, 2H-pyranyl, benzofuranyl, isobenzofuranyl, dibenzofuranyl, phenoxythiinyl, pyrrolyl, imidazolyl, pyrazolyl, pyridyl, bipyridyl, triazinyl, pyrimidinyl, pyrazinyl, pyridazinyl, indolizinyl, isoindolyl, indolyl, indazolyl, purinyl, quinolizinyl, quinolyl, isoquinolyl, phthalazinyl, naphthyridinyl, quinoxalinyl, quinazolinyl, cinnolinyl, pteridinyl, carbazolyl, carbolinyl, benzotriazolyl, benzoxazolyl, phenanthridinyl, acridinyl, perimidinyl, phenanthrolinyl, phenazinyl, isothiazolyl, phenothiazinyl, furazanyl or phenoxazinyl.

C₁-C₆Alkyl as R, R₁, R₂, R₃, R₄, R₉, R₁₀, R₁₁, R₁₂, R₁₆, R₁₇, R₁₈, R₂₀, R₂₁ and R₂₂ and as a substituent in R₁₉, R₂₀, R₂₁ and R₂₂ each independently of the others is e.g. methyl, ethyl, n-propyl, isopropyl, n-butyl, sec-butyl, tert-butyl, isobutyl, heptyl or hexyl.

R, R₁, R₂, R₁₀, R₁₁ and R₁₂ as C₆-C₂₄aryl are each independently of the others e.g. phenyl or naphthyl.

R₃ and R₄ as C₆-C₁₂aryl are each independently of the other e.g. phenyl or naphthyl.

R₅ as C₁-C₄alkyl is methyl, ethyl, n-propyl, isopropyl, n-butyl, sec-butyl, tert-butyl or isobutyl, preferably methyl or ethyl and especially methyl.

C₁-C₃Alkyl as R₆, R₇ and R₈ each independently of the others is methyl, ethyl, n-propyl or isopropyl.

As halogen there come into consideration for R, R₁, R₂, R₅, R₁₀, R₁₁and R₁₂ and as a substituent in R₃, R₇, R₈, R₁₉, R₂₀, R₂₁ and R₂₂ each independently of the others e.g. fluorine, chlorine or bromine, preferably chlorine or bromine and especially chlorine.

W as an aryl radical is preferably a phenylene, diphenylene or naphthylene radical, those radicals being unsubstituted or substituted by one or more substituents such as, for example, the substituents X and/or Y defined below.

Preferred as W is a radical of formula 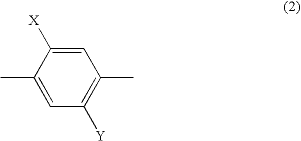
wherein
X is hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃⁻X⁺, or C₆-C₆-C₂₄aryl unsubstituted or mono- or poly-substituted by R₅, and
Y is hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃⁻X⁺, or C₆-C₂₄aryl unsubstituted or mono- or poly-substituted by R₅.

Ar₁ as an aryl radical is especially a phenyl or naphthyl radical, such radicals being unsubstituted or substituted by one or more substituents R₈.

Preferred as Ar₁ is a radical of formula 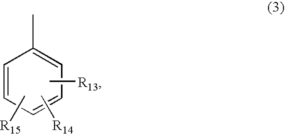
wherein
R₁₃, R₁₄ and R₁₅ are each independently of the others hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃R₄, SO₃⁻X⁺, or C₆-C₁₂aryl unsubstituted or mono- or poly-substituted by R₅.

Preferred as Ar₁₁ is a radical of formula 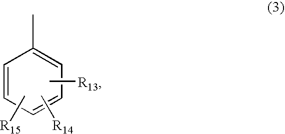
wherein
R₁₃, R₁₄ and R₁₅ are each independently of the others hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃R₄, SO₃⁻X⁺, or C₆-C₁₂aryl unsubstituted or mono- or poly-substituted by R₅.

In a preferred embodiment of the bisazoquinolone pigments of formula (1) according to the invention, R₁ and R₂ are each independently of the other hydrogen, C₁-C₃alkyl, C₁-C₃alkoxy, chlorine, COOR₅, NR₄COR₃, COO⁻X⁺ or SO₃⁻X⁺, R₅ is hydrogen or C₁-C₃alkyl, and X⁺ is a cation Na⁺, Mg⁺⁺_{1/2}, Ca⁺⁺_{1/2}, Sr⁺⁺_{1/2}, Ba⁺⁺_{1/2} or [NR₉R₁₀R₁₁R₁₂]⁺ wherein R₉, R₁₀, R₁₁ and R₁₂ are each independently of the others hydrogen, C₁-C₆alkyl, phenyl unsubstituted or mono- or poly-substituted by C₁-C₃alkyl, halogen, nitro, OR₇ or by N(R₇)₂, or benzyl unsubstituted or mono- or poly-substituted by C₁-C₃alkyl, halogen, nitro, OR₇ or by N(R₇)₂.

In a further preferred embodiment of the bisazoquinolone pigments of formula (1) according to the invention, R₁ and R₂ are each independently of the other hydrogen, C₁-C₂alkyl, C₁-C₂alkoxy, chlorine, COOR₅, NR₄COR₃, COO⁻X⁺ or SO₃⁻X⁺, R₅ is hydrogen or C₁-C₂alkyl, and X⁺ is a cation Na⁺, Mg⁺⁺_{1/2}, Ca⁺⁺_{1/2}, Sr⁺⁺_{1/2}, Ba⁺⁺_{1/2} or [NR₉R₁₀R₁₁R₁₂]⁺ wherein R₉, R₁₀,R₁₁ and R₁₂ are each independently of the others hydrogen, C₁-C₆alkyl, phenyl unsubstituted or mono- or poly-substituted by C₁-C₂alkyl and/or by halogen, or benzyl unsubstituted or mono- or poly-substituted by C₁-C₂alkyl and/or by halogen.

In a further, likewise preferred, embodiment of the bisazoquinolone pigments of formula (1) according to the invention, R₁ and R₂ are each independently of the other hydrogen, C₁-C₂alkyl, C₁-C₂alkoxy, chlorine, COOR₅ or NR₄COR₃ and R₅ is hydrogen or C₁-C₂alkyl.

Special preference is given to bisazoquinolone pigments of formula 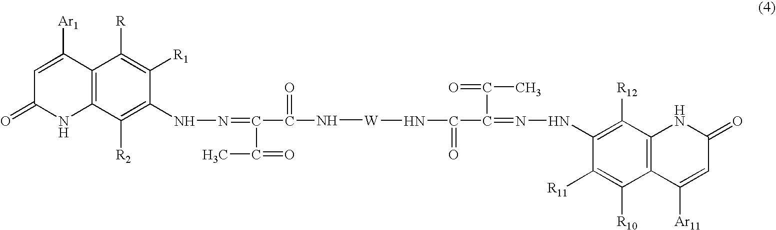
wherein R, R₁, R₂, R₁₀, R₁₁, R₁₂, Ar₁, Ar₁₁ and W have the definitions and preferred meanings indicated above.

Very special preference is given to bisazoquinolone pigments of formula 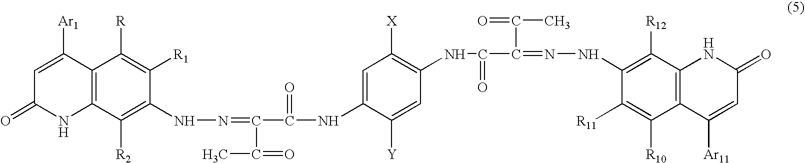
wherein R, R₁, R₂, R₁₀, R₁₁, R₁₂, Ar₁, Ar₁₁, X and Y have the definitions and preferred meanings given above.

The bisazoquinolone pigments listed in Table 1 below are important:

TABLE 1

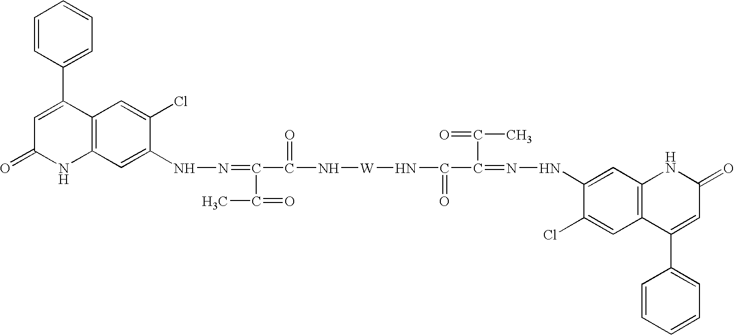

| Formula No. | W | Shade |
|---|---|---|
| 100 | 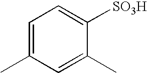 | yellow |
| 100a | 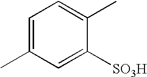 | yellow |
| 101 | 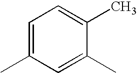 | yellow |
| 101a | 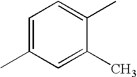 | yellow |
| 102 | 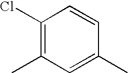 | greenish yellow |
| 103 | 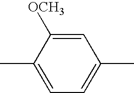 | yellow |
| 104 | 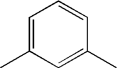 | yellow |
| 105 | 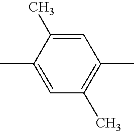 | reddish yellow |
| 106 | 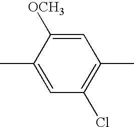 | yellow |
| 107 | 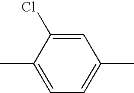 | greenish yellow |
| 108 | 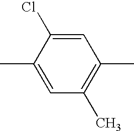 | greenish yellow |
| 109 | 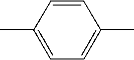 | greenish yellow |
| 110 | 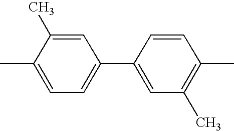 | greenish yellow |
| 111 | 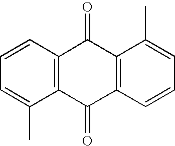 | orange |
| 112 | 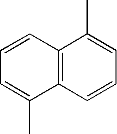 | greenish yellow |

and the bisazoquinolone pigment of formula

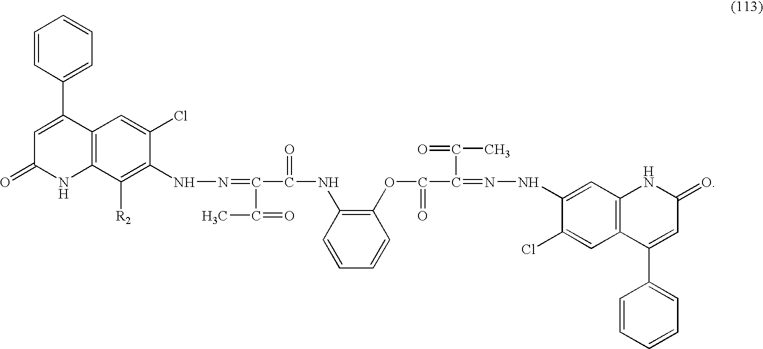

The present invention relates also to a process for the preparation of bisazoquinolone pigments of formula (1), which process comprises reacting, for example, 2 mol of a compound of formula 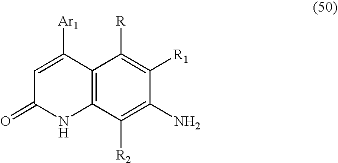
or 1 mol of a compound of formula (50) and 1 mol of a compound of formula 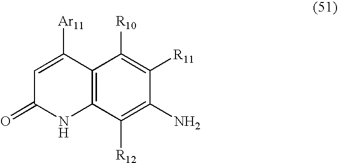
in an acidic medium at elevated temperature with 1 mol of a compound of formula 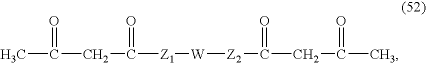
and then adding tert-butyl nitrite to form a compound of formula (1),
or
treating, for example, 2 mol of a compound of formula (50) or 1 mol of a compound of formula (50) and 1 mol of a compound of formula (51) at a temperature of from 0 to 10° C. with 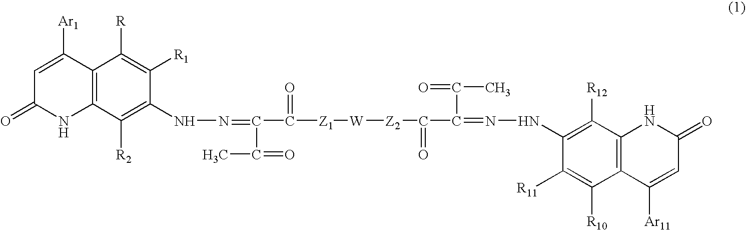 an acid and then converting into the corresponding diazonium salt with a stoichiometric amount of sodium nitrite solution and reacting the diazonium salt in water at a pH of from 4 to 10 with 1 mol of a compound of formula (52) to form a compound of formula (1), wherein W, Ar₁, Ar₁₁, R, R₁, R₂, R₁₀, R₁₁, R₁₂, Z₁ and Z₂ have the definitions and preferred meanings indicated above.

The compounds of formulae (50) and (51) can be obtained, for example, in accordance with the method described in JP-A-2001-287466 or in Journal of Chemical and Engineering Data, Vol. 13 No. 3, July 1968. The compounds of formula (52) are known or can be prepared analogously to generally known methods.

The bisazoquinolone pigments of formula (1) according to the invention are suitable as colorants for the colouring of high molecular weight material.

The high molecular weight material can be organic or inorganic and may be a synthetic and/or natural material. It may be, for example, a natural resin or a drying oil, rubber or casein, or a modified natural material such as chlorinated rubber, an oil-modified alkyd resin, viscose, a cellulose ether or ester, such as ethyl cellulose, cellulose acetate, propionate or butyrate, cellulose acetobutyrate as well as nitrocellulose, but especially a totally synthetic organic polymer (thermosetting plastics and thermoplastics), as can be obtained by polymerisation, for example by polycondensation or polyaddition. The polymer class includes, for example, polyolefins, such as polyethylene, polypropylene, polyisobutylene, also substituted polyolefins, such as polymerisation products of monomers such as vinyl chloride, vinyl acetate, styrene, acrylonitrile, acrylic acid esters, methacrylic acid esters, fluoropolymers, for example polyfluoroethylene, polytrifluorochloroethylene or tetrafluoroethylene/hexafluoropropylene mixed polymers, as well as copolymers of the mentioned monomers, especially ABS (acrylonitrile/butadiene/styrene) or EVA (ethylene/vinyl acetate). From the group of the polyaddition and polycondensation resins it is possible to use, for example, condensation products of formaldehyde with phenols, the so-called phenoplasts, and condensation products of formaldehyde and urea or thiourea, and also melamine, the so-called aminoplasts, also the polyesters used as surface-coating resins, either saturated, such as alkyd resins, or unsaturated, such as maleic resins, also linear polyesters, polyamides, polyurethanes, polycarbonates, polyphenylene oxides or silicones, and silicone resins.

The mentioned high molecular weight compounds may be present individually or in mixtures, in the form of plastic compositions or melts, which may optionally be spun to form fibres.

They may also be present in the form of their monomers or in the polymerised state in dissolved form as film-forming agents or binders for surface coatings, paints or printing inks, such as boiled linseed oil, nitrocellulose, alkyd resins, melamine resins, urea-formaldehyde resins or acrylic resins.

Pigmenting of the high molecular weight organic materials with the bisazoquinolone pigments according to the invention is carried out, for example, by adding the pigment, optionally in the form of a masterbatch, to the substrates using rolling mills or mixing or grinding apparatus. The pigmented material is then generally brought into the desired final form by methods known per se, such as calendering, compression moulding, extrusion, spread-coating, casting or by injection moulding. It is often desirable, in order to produce non-rigid mouldings or to reduce their brittleness, to incorporate so-called plasticisers into the high molecular weight compounds prior to shaping. There may be used as plasticisers, for example, esters of phosphoric acid, phthalic acid or sebacic acid. In the process according to the invention the plasticisers may be incorporated into the polymers before or after the incorporation of the pigment colorant. It is also possible, in order to achieve different shades of colour, to add to the high molecular weight organic materials, in addition to the bisazoquinolone pigments of formula (1), also fillers or other colour-imparting constituents, such as white, coloured or black pigments as well as special-effect pigments, in each case in the desired amount.

For the pigmenting of surface coatings and printing inks, the high molecular weight organic materials and the bisazoquinolone pigments of formula (1), optionally together with additives such as fillers, other pigments, for example TiO₂, siccatives or plasticisers, are generally finely dispersed or dissolved in an organic and/or aqueous solvent or solvent mixture. The procedure may be such that the individual components are dispersed or dissolved separately, or several are dispersed or dissolved together, and only then are all the components combined.

Based on the high molecular weight material to be pigmented, the bisazoquinolone pigments according to the invention can be used in an amount of from 0.01 to 30% by weight, preferably from 0.1 to 10% by weight.

Accordingly, the present invention relates also to the use of the bisazoquinolone pigments of formula (1) according to the invention in the colouring of high molecular weight material, especially organic high molecular weight material, which includes the use as a colorant for plastics in any form, for example in the form of fibres, surface coatings or printing inks.

The bisazoquinolone pigments of formula (1) according to the invention are especially suitable for the mass-colouring of polyvinyl chloride and, especially, polyolefins, such as polyethylene and polypropylene, as well as of aqueous and/or solvent-based lacquers, for example automotive lacquers, and also powder coatings, printing inks and paints.

The resulting colorations, for example in plastics, fibres, surface coatings or prints, are distinguished by a high purity of shade, high tinctorial strength, good fastness to overspraying and to migration, and especially by good fastness to heat, to light and to weathering, for example in full-shade or reduction-with-white applications.

The bisazoquinolone pigments of formula (1) according to the invention are further distinguished by good dispersibility, by good rheological behaviour and by colorations obtained therewith having a high gloss.

The bisazoquinolone pigments of formula (1) according to the invention are also suitable as colorants for the production of colour filters, especially for visible light in the range from 400 to 700 nm, for liquid crystal displays (LCD) or charge combined devices (CCD).

The production of colour filters by sequential application of a red, blue and green pigment to a suitable substrate, for example amorphous silicon, is described in GB-A 2 182 165. The colour filters can be coated, for example, using inks, especially printing inks, comprising the bisazoquinolone pigments according to the invention, or, for example, by mixing the bisazoquinolone pigments according to the invention with chemically, thermally or photolytically structurable high molecular weight material. The further production can be carried out, for example, analogously to EP-A 654 711 by application to a substrate, such as an LCD, subsequent photostructuring and development. A further document which describes the production of colour filters is US-A-5 624 467.

The colour filters produced with the pigments according to the invention for liquid crystal displays (LCD) exhibit an improved transmission window between 500 and 600 nm in comparison with colour filters known hitherto, and they are therefore distinguished by high transmission of the green colour points. The colour filters produced with the pigments according to the invention exhibit increased absorption at 400 nm in comparison with the colour filters known hitherto, which advantageously yields a more yellowish green.

The following Examples serve to illustrate the invention. In the Examples, unless otherwise indicated, parts are parts by weight and percentages are percent by weight. The temperatures are given in degrees Celsius. The relationship between parts by weight and parts by volume is the same as that between grams and cubic centimetres.

### EXAMPLE 1

A suspension of 1.36 g (4 mmol) of 1,4-bisacetoacetamido-2-methoxy-5chlorobenzene and 2.18 g (8 mmol) of 7-amino-6-chlorophenylquinolin-2-one in 60.0 ml of acetic acid and 70 ml of diglyme is heated at 80° C. for 60 minutes and then cooled to 40° C. 5.5 ml (8 mmol) of a 15% solution of tert-butyl nitrite in acetic acid are added dropwise to the resulting reddish-brown solution within a period of 80 minutes, the pigment being precipitated in the form of an orange-coloured solid.

The reaction mixture is heated at 80° C. for 6 hours and then maintained at 50° C. for 16 hours. The crude pigment is filtered while still warm, washed with copious amounts of water and dried in vacuo for 20 hours at 110° C.

3.03 g (3.3 mmol, yield 83% of theory) of a compound of formula 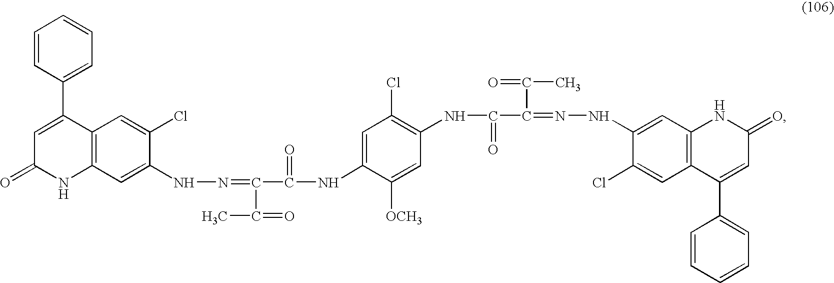
are obtained, which in high molecular weight organic material yields yellow colorations having very good fastness to weathering.

### EXAMPLE 2

10.82 g (40 mmol) of 7-amino-6-chloro-4-phenyl-2-quinolone in 200 ml of 100% acetic acid are heated to 100° C. within a period of 20 minutes, a clear pink solution being formed. The solution is cooled to 10° C. and 26.5 ml of 37% aqueous hydrochloric acid are stirred in, the internal temperature being maintained at 10° C. by cooling. The hydrochloride formed is obtained in the form of a brownish-yellow precipitate. Within a period of 10 minutes, 10 ml of 4N aqueous sodium nitrite solution (40 mmol) are then metered in, with effective cooling, in such a manner that the temperature at the end of the addition is still 8° C. After a further 20 minutes with cooling—the temperature has then decreased to 5° C.—the diazonium salt is present in the form of a clear, yellow solution. The solution is to be reacted further immediately in order to avoid possible decomposition.

In the meantime, 5.52 g (20 mmol) of 1,4-bis(acetoacetylamino)benzene are stirred at room temperature in 280 ml of deionised water and, with 8 ml of 30% aqueous sodium hydroxide solution, dissolved with intensive stirring. The solution, which is still slightly cloudy, is clarified by filtration over a paper filter with a small amount of HYFLO (kieselguhr; filtration aid): clear colourless solution, the pH is 12.9. By adding 20 ml of 50% (vol.) aqueous acetic acid, the solution is adjusted to pH 4.5 and is immediately cooled to 1° C. The above diazonium salt solution, which has been freshly prepared and cooled to 0° C., is then stirred into the resulting fine suspension of the coupling component within a period of 1 hour and 50 minutes, the pH value of the reaction solution being maintained constant at a value of 4.5 by controlled addition of 30% aqueous sodium hydroxide solution (total consumption: 180.7 ml) and the temperature being maintained at below 5° C. by cooling. The orange suspension is heated to 85° C., stirred at that temperature for one hour and filtered over a hard paper filter while still hot. The press cake is washed with deionised water until the filtrate is pH-neutral. The still moist crude product is stirred in 320 ml of dimethylacetamide, heated to 125° C. and stirred at that temperature for a further 14 hours. The yellowish-range suspension is filtered over a hard paper filter while still hot and washed with 200 ml of dimethylacetamide, then with 200 ml of methanol and finally with 200 ml of deionised water. After drying for 20 hours at 110° C. in vacuo, there are obtained 14.96 g (89% of theory) of a compound of formula 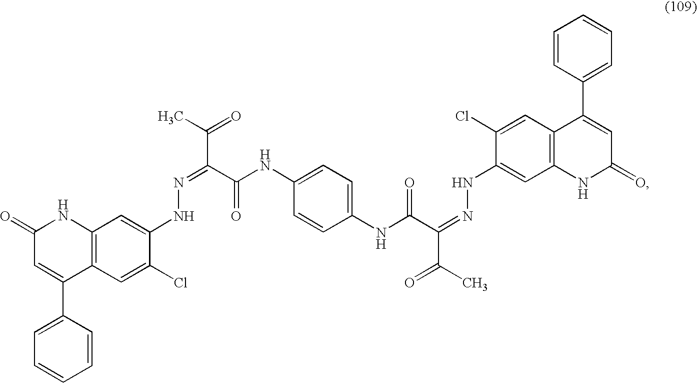
which in high molecular weight organic material yields greenish-yellow colorations having very good fastness to weathering.

### EXAMPLE 3

5.41 g (20 mmol) of 7-amino-6-chloro-4-phenyl-2-quinolone are stirred in 160 ml of deionised water at room temperature, and 100 ml of 98% acetic acid and 7 ml of 37% aqueous hydrochloric acid are added. The mixture is heated to 80° C. and stirred for a further one hour at that temperature. The white suspension of the resulting hydrochloride is then cooled to 0° C. and, within a period of 5 minutes, 5 ml of 4N aqueous sodium nitrite solution (20 mmol) are stirred in, the internal temperature being maintained constant at 0° C. The diazonium salt so prepared in the form of a light-green suspension is to be reacted further immediately in order to avoid possible decomposition.

In the meantime, 3.4 g (10 mmol) of 1,4-bis(acetoacetylamino)-2-chloro-5-methoxybenzene are stirred at room temperature in 200 ml of deionised water and, with 2 ml of 30% aqueous sodium hydroxide solution, dissolved with intensive stirring. The clear solution is cooled to 2° C. (measured pH value 12.4) and adjusted to pH 4.5 with 5.5 ml of 50% (vol.) aqueous acetic acid. The above diazonium salt suspension, which has been freshly prepared and cooled to 0° C., is then stirred into the resulting fine grey suspension of the coupling component within a period of 50 minutes, the pH of the reaction solution being maintained constant at a value of 4.5 by controlled addition of 30% aqueous sodium hydroxide solution (total consumption: 39.8 ml) and the temperature of the reaction mass being maintained at below 3° C. by cooling. The yellowish-orange suspension is filtered over a hard paper filter and the press cake is washed with deionised water until the filtrate is pH-neutral. The still moist crude product is stirred in 180 ml of dimethylacetamide and heated to an internal temperature of 130° C.; initially a small amount of the water/solvent mixture needs to be distilled off by way of a curved distillation tube in order that the required temperature is reached. Stirring is continued for 16 hours at 130° C., the yellowish-orange suspension is filtered over a hard paper filter while still hot and washed with 200 ml of methanol and then with 100 ml of deionised water. After drying for 20 hours at 110° C. in vacuo there are obtained 2.25 g (25% of theory) of a compound of formula 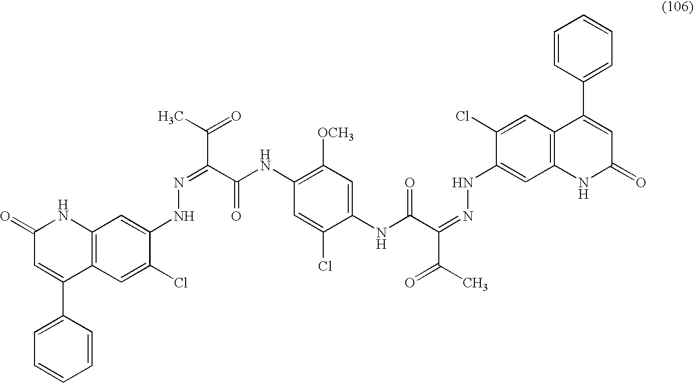
which in high molecular weight organic material yields yellow colorations having very good fastness to weathering.

### APPLICATION EXAMPLE 1

### Use in the Mass-Colouring of Plastics

0.9 g of the pigment of formula (106) is mixed with 67 g of polyvinyl chloride, 33 g of dioctyl phthalate, 2 g of dibutyltin dilaurate and 2 g of titanium dioxide and processed to a thin film on a roller apparatus for 15 minutes at 160° C. The yellow PVC film so obtained is tinctorially strong and fast to light.

### APPLICATION EXAMPLE 2

### Use in an Alkyd-Melamine Stoving Lacquer

A mixture of 460 g of steatite spheres having a diameter of 8 mm, an alkyd lacquer consisting of 58.7 g of alkyd resin ®Alkydal F 310 (Bayer AG), 60% in xylene, 58.7 g of alkyd resin ®Alkydal F 32 (Bayer AG), 60% in xylene, 2.0 g of ®Silikonöl A (Bayer AG), 1% in xylene, 4.0 g of n-butanol, 4.0 g of Dowanol, 15 g of xylene, 5.6 g of dispersing agent ®Disperbyk D-160 (BYK-Chemie) and also 36.0 g of the pigment of formula (106) is dispersed in a glass bottle having a twist-off cap for 72 hours on a tumbler. After addition of 24.0 g of the melamine component ®Cymel 327 (Cyanamid), 90% in xylene, dispersion is continued for a further one hour on the tumbler. The steatite spheres are then separated off. The resulting colour lake paste is applied to ®Milar transparency films and then stoved for 30 minutes at 130° C. (lacquer layer thickness 50 μm). A coloration having excellent coloristic and rheological properties is obtained.

### APPLICATION EXAMPLE 3

### Preparation of an Intaglio/Flexographic Printing Ink

21 g of the pigment of formula (106)
20 g of clear lacquer consisting of
   20 g of nitrocellulose type A
   4 g of dioctyl phthalate
   56 g of ethanol and
   20 g of ethyl acetate and
259 of ethanol
   are dispersed for 30 minutes by means of a high-speed stirrer (dissolver at 15 m/s). 40 g of the clear lacquer described above are then added to the batch, and dispersion is carried out for a further 5 minutes with the dissolver. The grinding charge is introduced into a bead mill by means of a pump with coarse filtering, and is finely dispersed therein. Extraordinary transparency/gloss properties are obtained with this printing ink both in intaglio/flexographic printing and in offset printing.

### APPLICATION EXAMPLE 4

### Production of a Colour Filter for Liquid Crystal Displays (LCD)

In a 100 ml glass vessel containing 83.3 g of zircon ceramic spheres, 3.8 g of the pigment of formula (106), 0.28 g of Solsperse® 5000, 4.10 g of Disperbyk® 161 (dispersing agent, 30% solution of a high molecular weight block copolymer containing groups having affinity for pigments, in n-butyl acetate/1-methoxy-2-propyl acetate 1:6, BYK Chemie) and 14.62 g of propylene glycol monomethyl ether acetate (MPA, CAS Reg. N^{Q} 108-65-6) are stirred by means of a Dispermat for 10 minutes at 1000 rev/min and for 180 minutes at 3000 rev/min at 23° C. After addition of 4.01 g of an acrylate polymer binder (35% solution in MPA), stirring is carried out for 30 minutes at 3000 rev/min at room temperature. After separation of the spheres, the dispersion is diluted with the same weight of MPA.

A glass substrate (Corning type 1737-F) is coated with the dispersion on a centrifugal lacquer coating apparatus and centrifuged off for 30 seconds at 1000 rev/min. The layer is dried for 2 minutes at 100° C. and for 5 minutes at 200° C. on a hotplate. The layer thickness achieved is 0.4 μm.

### APPLICATION EXAMPLE 5

A mixture of 1.5 g of the pigment of formula (106), 1.0 g of a commercially available antioxidant (®Irganox1010, Ciba Specialty Chemicals AG) and 1000 g of polyethylene HD granules (®Vestolen 60-16, Huels) is pre-mixed for 15 minutes in a glass bottle on a roller bench. The mixture is then extruded on a single-screw extruder in two passes, and the granules so obtained are compressed to sheets on an injection-moulding machine (Ferromatik Aarburg 200) for 5 minutes at 200° C., 5 minutes at 240° C., 5 minutes at 260° C., 5 minutes at 280° C. and 5 minutes at 300° C.

The sheets exhibit tinctorially strong yellow shades with good stability properties.

If, in the above Application Examples, instead of the pigment of formula (106) there is used a pigment of formula (100) to (105), (107) to (112) listed in Table 1 or the pigment of formula (113), the colorations obtained likewise exhibit good properties.

1. A bisazoquinolone pigment which, in one of the tautomeric forms thereof, corresponds to formula 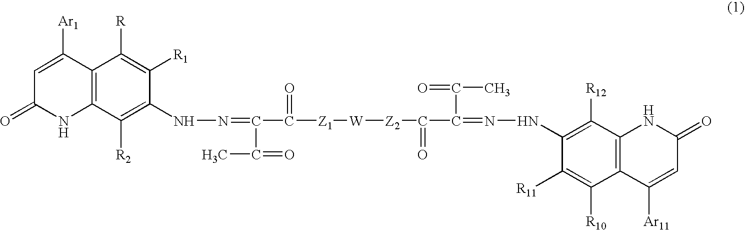
wherein
W is the radical of an unsubstituted or substituted C₆-C₂₄aryl or the radical of an unsubstituted or substituted heteroaryl,
Ar₁ is unsubstituted or substituted C₆-C₂₄aryl or unsubstituted or substituted heteroaryl,
Ar₁₁ is unsubstituted or substituted C₆-C₂₄aryl or unsubstituted or substituted heteroaryl,
R, R₁, R₂, R₁₀, R₁₁ and R₁₂ are each independently of the others hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃⁻X⁺, or C₆-C₂₄aryl unsubstituted or mono- or poly-substituted by R₅,
R₃ is C₁-C₆alkyl, or C₆-C₁₂aryl unsubstituted or mono- or poly-substituted by halogen, hydroxy, OR₇, cyano, nitro, SR₇, NR₆R₇, COOR₇, CONR₆R₇, NR₆COR₇, NR₆COOR₇, COO⁻X⁺, COR₄, OR₄, SO₂R₇, SO₂NR₆R₇, SO₃⁻X⁺ or by SO₃R₇,
R₄ is hydrogen or has the same meanings as R₃,
R₅ is hydrogen, C₁-C₄alkyl, halogen, nitro, NR₇R₈ or OR₇,
R₆ is hydrogen or C₁-C₃alkyl,
R₇ and R₈ are each independently of the other hydrogen, C₁-C₃alkyl, phenyl unsubstituted or mono- or poly-substituted by halogen, nitro, OR₅ or by NR₁₆R₁₇, or benzyl unsubstituted or mono- or poly-substituted by halogen, nitro, OR₅ or by NR₁₆R₁₇, and X⁺ is a cation H⁺, Li⁺, Na⁺, K⁺, Mg⁺⁺_{1/2}, Ca⁺⁺_{1/2}, Sr⁺⁺_{1/2}, Ba⁺⁺_{1/2}, Cu⁺, Cu⁺⁺_{1/2}, Zn⁺⁺_{1/2}, Mn⁺⁺_{1/2}, Al⁺⁺⁺_{1/3} or [NR₁₉R₂₀R₂₁R₂₂]⁺ wherein R₁₉, R₂₀, R₂ and R₂₂ are each independently of the others hydrogen, C₁-C₆alkyl, phenyl unsubstituted or mono- or poly- substituted by C₁-C₆alkyl, halogen, nitro, OR₅ or by NR₁₆R₁₇, or benzyl unsubstituted or mono- or poly-substituted by C₁-C₆alkyl, halogen, nitro, OR₅ or by NR₁₆R₁₇,
R₁₆ and R₁₇ are each independently of the other hydrogen or C₁-C₆alkyl,
Z₁, is —NH— or —O—, and
Z₂ is —NH— or —O—.

2. A bisazoquinolone pigment according to claim 1, wherein W is a radical of formula 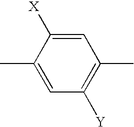
X is hydrogen, C₁-C₆alkyl, halogen, cyano, CF₃, nitro, NR₃R₄, COOR₄, NR₄COR₃, COO^{-X}⁺, COR₄, OR₄, SR₃, SO₂R₃, SO₂NR₃R₄, SO₃^{-X}⁺, or C₆-C₂₄aryl unsubstituted or mono- or poly-substituted by R₅, and
Y is hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃⁻X⁺, or C₆-C₂₄aryl unsubstituted or mono- or poly-substituted by R₅.

3. A bisazoquinolone pigment according to claim 1, wherein Ar₁ is a radical of formula 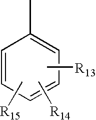
R₁₃, R₁₄ and R₁₅ are each independently of the others hydrogen, C₁-C₆alkyl, halogen, cyano, CF₃, nitro, NR₃R₄, COOR₄, NR₄COR₃, COO^{-X}⁺, COR₄, OR₄, SR₃, SO₂R₃, SO₂NR₃R₄, SO₃R₄, SO₃^{-X}⁺, or C₆-C₁₂aryl unsubstituted or mono- or poly-substituted by R₅.

4. A bisazoquinolone pigment according to claim 1, wherein Ar₁₁ is a radical of formula 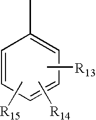
and
R₁₃, R₁₄ and R₁₅ are each independently of the others hydrogen, C₁-C₆alkyl, halogen, cyano, CF₃, nitro, NR₃R₄, COOR₄, NR₄COR₃, COO^{-X}⁺, COR₄, OR₄, SR₃, SO₂R₃, SO₂NR₃R₄, SO₃R₄, SO₃^{-X}⁺, or C₆-C₁₂aryl unsubstituted or mono- or poly-substituted by R₅.

5. A process for the preparation of a bisazoquinolone pigment according to claim 1, which process comprises reacting 2 mol of a compound of formula 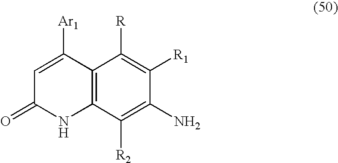
or 1 mol of a compound of formula (50) and 1 mol of a compound of formula 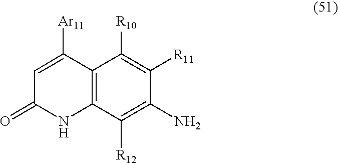
in an acidic medium at elevated temperature with 1 mol of a compound of formula 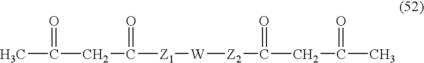
and then adding tert-butyl nitrite to form a compound of formula (1),
or treating, for example, 2 mol of a compound of formula (50) or 1 mol of a compound of formula (50) and 1 mol of a compound of formula (51) at a temperature of from 0 to 10° C. with an acid and then converting into the corresponding diazonium salt with a stoichiometric amount of sodium nitrite solution and reacting the diazonium salt in water at a pH of from 4 to 10 with 1 mol of a compound of formula (52) to form a compound of formula (1),
wherein W, Ar₁, Ar₁₁, R, R₁, R₂, R₁₀, R₁₁, R₁₂, Z₁ and Z₂ have the definitions and preferred meanings indicated above.

6. A high molecular weight material comprising as a colorant a bisazoquinolone pigment according to claim 1.

7. A plastic, surface coating or printing ink comprising as a colorant a bisazoquinolone pigment according to claim 1.

8. A color filter comprising as a colorant bisazoquinolone pigment according to claim 1.

9. A bisazoquinolone pigment according to claim 2, wherein Ar₁ is a radical of formula 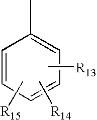
R₁₃, R₁₄ and R₁₅ are each independently of the others hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃R₄, SO₃⁻X⁺, or C₆-C₁₂aryl unsubstituted or mono- or poly-substituted by R₅.

10. A bisazoquinolone pigment according to claim 2, wherein Ar₁₁, is a radical of formula 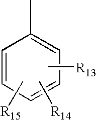
and
R₁₃, R₁₄ and R₁₅ are each independently of the others hydrogen, C₁-C₆alkyl, halogen, cyano, CF₃, nitro, NR₃R₄, COOR₄, NR₄COR₃, COO⁻X⁺, COR₄, OR₄, SR₃, SO₂R₃, SO₂NR₃R₄, SO₃R₄, SO₃^{-X}⁺, or C₆-C₁₂aryl unsubstituted or mono- or poly-substituted by R₅.
